(19) United States
(12) Patent Application Publication
(10) Pub. No.: US 2026/0325203 A1
(43) Pub. Date: Oct. 22, 2026

(54) BALLOON CATHETER WITH ENHANCED SCORING CAPABILITY

(71) Applicant: BARD PERIPHERAL VASCULAR, INC., Franklin Lakes, NJ (US)

(72) Inventor: Matthew R. Casiraro, Tempe, AZ (US)

(21) Appl. No.: 19/104,602

(22) PCT Filed: Aug. 18, 2022
(86) PCT No.: PCT/US2022/040742

§ 371 Date: Feb. 18, 2025

(51) Int. Cl. A61B 18/14; A61B 17/00; A61B 18/00; A61B 18/12; A61M 25/10

(52) U.S. Cl. CPC A61B 18/1492; A61B 18/1206; A61M 25/1002; A61B 2017/00557; A61B 2017/0088; A61B 2018/00077; A61B 2018/00083; A61B 2018/0016; A61B 2018/0022; A61B 2018/00577; A61B 2018/00613; A61B 2018/144; A61B 2018/1467; A61M 2025/1086

(57) ABSTRACT

An apparatus for performing a medical procedure on a structure, such as a lesion, in a vessel. The apparatus includes an inflatable balloon and at least one conductive wire extending along the balloon and adapted for scoring the structure when the inflatable balloon is inflated. A generator is also provided for supplying electrical energy, such as radiofrequency energy, to the at least one conductive wire. An insulator, such as a rigid ceramic material, may be provided between the at least one conductive wire and an outer surface of the balloon. The generator may selectively apply electrical energy to some of a plurality of conductive wires. Related methods are also provided.

### BACKGROUND

Dilatation catheters with expandable elements or balloons are of-ten used to treat lesions in vessels. However, difficulties are encountered in navigating tortuous anatomy and safely crossing very tight lesions. Moreover, some lesions are difficult to dilate using just a balloon, and require the application of a focused force, such as provided by a scoring wire to dilate the lesion, in order to achieve ablation.

In certain applications, such as in a heavily calcified vessel, the scoring wire may sometimes be limited effectiveness, since it can only apply a certain amount of focused force. Adding inflation pressure to the balloon in an effort to increase the amount of radial force not only risks potential bursting of the balloon as a result of the increased internal pressure, but also risks dissecting the balloon as a result of contact with the scoring element.

Accordingly, a need is identified for a balloon catheter with an enhanced scoring wire. The scoring wire would be better able to achieve scoring effect without the need for increasing the pressure applied to the balloon. The amount of ablation provided would also be controllable in-dependent of the pressurization status of the balloon. Improved treatment outcomes could thus be achieved with a potential reduction in procedure time.

### SUMMARY

An object of the disclosure is to provide a balloon catheter with an enhanced scoring wire. The scoring wire would be better able to achieve scoring effect, such as during percutaneous transluminal angioplasty, without the need for increasing the pressure applied to the balloon. The amount of ablation provided by the scoring wire would also be controllable by the application of electrical energy, and thus operate independently of the pressurization status of the balloon. Improved treatment outcomes could thus be achieved with a potential reduction in procedure time.

According to one aspect of the disclosure, an apparatus for performing a medical procedure on a structure in a vessel is provided. The apparatus comprises an inflatable balloon and at least one conductive wire extending along the balloon and adapted for scoring the structure when the inflatable balloon is inflated. A generator is provided for supplying electrical energy to the at least one conductive wire.

In one embodiment, an insulator is provided between an outer surface of the inflatable balloon and the at least one conductive wire. The insulator may comprise a rigid insulating material, such as a ceramic. Alternatively or additionally, the insulator may comprise a cradle for receiving the at least one conductive wire.

The at least one conductive wire may comprise a plurality of conductive wires, such as for example four wires spaced equidistantly around the circumference of the balloon. The generator may be adapted to apply electrical energy selectively to one or more of the plurality of conductive wires.

A shaft may support the inflatable balloon and include a lumen for receiving a portion of the at least one conductive wire proximal of the balloon. The balloon may comprise a tapered proximal section and a barrel section distal thereof, with a portion of the at least one conductive wire along the tapered proximal section being insulated.

The generator may be adapted to supply electrical energy so as to perform RF ablation. Alternatively, the generator may be adapted to perform irreversible electroporation.

According to a further aspect of the disclosure, an apparatus for performing a medical procedure on an anatomical structure is provided. The apparatus comprises an inflatable balloon and at least one conductive wire extending along the balloon and adapted for scoring the structure when the inflatable balloon is inflated. A rigid member is provided between an outer surface of the inflatable balloon and the at least one conductive wire.

In one embodiment, the rigid member comprises an insulator. A generator may also be provided for supplying electrical energy to the at least one conductive wire. The generator may be adapted to perform RF ablation and/or irreversible electroporation (IRE).

The apparatus may further include a shaft supporting the inflatable balloon, the shaft including a lumen for receiving a portion of the at least one conductive wire proximal of the balloon. The balloon may comprise a tapered proximal section and a barrel section distal thereof, a portion of the at least one conductive wire along the tapered proximal section being insulated. The rigid member may comprise a cradle for receiving the at least one conductive wire, which cradle may be formed of a ceramic material.

According to a further aspect of the disclosure, a method of performing a procedure on an anatomical structure is provided. The method comprises scoring the structure with a scoring wire extending along an inflatable balloon, and applying electrical energy to the scoring wire.

In one embodiment, the step of applying electrical energy comprises performing RF ablation. In another embodiment, the step of applying electrical energy comprises performing irreversible electroporation. The method may further include the step of providing an insulator for insulating the at least one scoring wire from an outer surface of the inflatable balloon.

The scoring balloon may include a plurality of scoring wires, and the step of applying electrical energy comprises applying electrical energy to each of the plurality of scoring wires simultaneously. Alternatively, the step of applying electrical energy comprises selectively applying electrical energy alternatively to the plurality of scoring wires.

Still a further aspect of the disclosure pertains to a method of performing a medical procedure on an anatomical structure. The method comprises scoring the structure with a scoring wire spaced from an outer surface of an inflatable balloon by a rigid member. The method may further include the step of applying electrical energy to the scoring wire.

### BRIEF DESCRIPTION OF THE DRAWINGS

The above and further advantages of the disclosure may be better understood by referring to the following description in conjunction with the accompanying drawings in which:

FIG. 1 illustrates a cutaway perspective view of a balloon catheter with enhanced scoring capability according to one aspect of the disclosure;

FIG. 2 is an end view of the balloon catheter of FIG. 1;

FIG. 3 is a cross-section taken along line 3-3 of FIG. 1;

FIG. 3A is an enlarged cross-sectional view of a single conductive wire, insulator forming a cradle for the wire, and a portion of the under-lying balloon;

FIG. 4 is a side-view of a balloon catheter with enhanced scoring capability including a proximal portion of the catheter;

FIG. 5 is an enlarged side view of the balloon catheter in use; and

FIGS. 6 and 7 are cutaway perspective and end views of an alter-native embodiment.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the items depicted in the drawings may be combined into a single function.

### DETAILED DESCRIPTION

In the following detailed description, numerous specific details are set forth to provide a thorough understanding of the present invention. The disclosed embodiments may be practiced without these specific details. In other instances, well-known methods, procedures, components, or structures may not have been described in detail so as not to obscure the present invention.

The principles and operation of the apparatus and methods of the disclosure may be better understood with reference to the drawings and accompanying descriptions. The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination.

With reference to FIG. 1, a catheter 10 is shown, which includes an inflatable medical balloon 12 attached to a distal end portion of a catheter shaft 14. The balloon 12 may be inflated by way of an inflation lumen L within the shaft 14 for delivering a fluid to an interior compartment I thereof. This lumen L may be formed by an outer tube 16 coaxial with an inner tube 18 forming a guidewire lumen G extending through the balloon 12 to a distal tip 20. Alternatively, a dual lumen approach could be used, where the lumens G, L are arranged side-by-side, as could a short exchange arrangement, where the shaft 14 is adapted to receive the guidewire at a port (not shown) adjacent to a proximal end of the balloon 12.

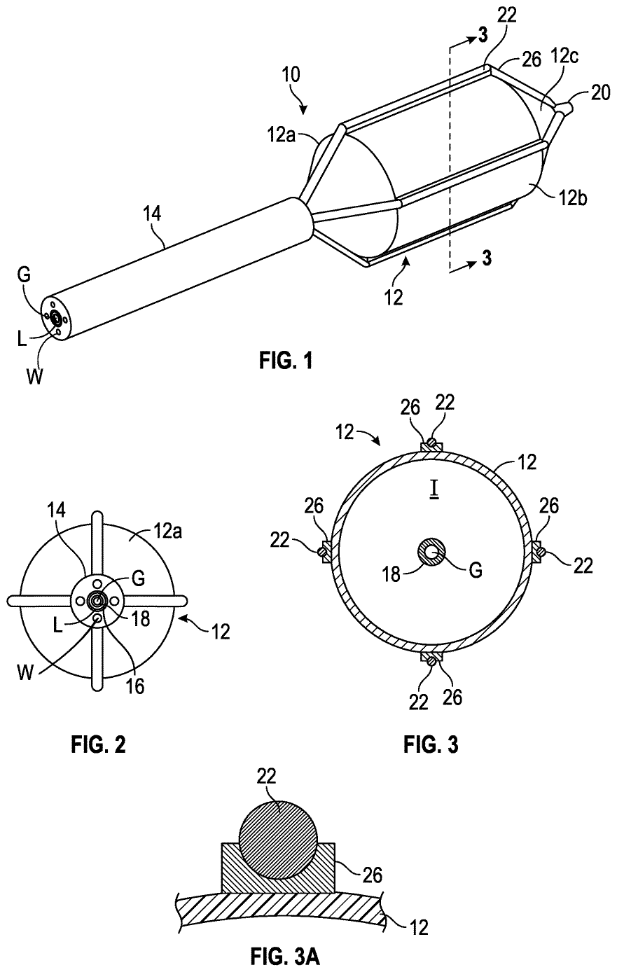

The balloon 12 is provided with one or more scoring elements, which are shown in the form of one or more wires 22. The wire(s) 22 may extend from a proximal end of the balloon comprising a proximal tapered cone 12a, along a center cylindrical or barrel section 12b, and along a distal tapered cone 12c to adjacent the tip 20. As can be understood from FIGS. 2-3, the present balloon 12 includes four such wires 22 spaced approximately equidistantly around the balloon circumference (i.e. 90 degrees apart), but more or fewer can be provided, and different spacing may be provided among the wires, as necessary or desired.

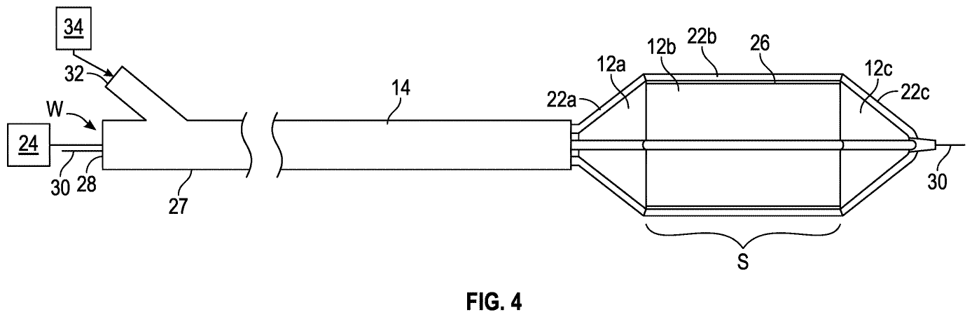

According to one aspect of the disclosure, the wire(s) 22 are adapted for both scoring and ablating an anatomical structure, such as a lesion (but other structures are discussed hereinbelow), using the application of energy from a source. This may be achieved by making the wire(s) 22 conductive at least along a portion of the balloon 12, such as a distal portion of each wire. As shown in FIG. 4, portions 22a of the wire(s) 22 proximal to the balloon 12 may enter the shaft 14 and extend through corresponding lumen(s) W to a proximal end. The distalmost ends of the distal portions 22c of the wire(s) may connect to the tip 20.

As indicated in FIG. 4, the wire(s) 22 may connect to a source in the form of a generator 24 for generating energy, such as electrical energy, in order for the wire(s) to have an ablative effect when brought into contact with tissue, such as upon inflation of the balloon 12. The generator 24 may be part of a hub 27 connected to the proximal end of the shaft 14, and may include a first port 28 for receiving a guidewire 30, and a second port 32 for receiving inflation fluid, such as from an indeflator 34. The generator 24 may also be distinct or separate from the hub 27, or may be adapted to releasably connect to it for selective use when enhanced scoring is desired.

In the case of plural wires 22, the arrangement may be such that the connection to the generator 24 causes electrical energy to be sup-plied to all wire(s) present simultaneously. Alternatively, the arrangement may be selective, such as by using a switch or the like, to allow energy to be delivered to certain of the wire(s) 22, but not other(s), in order to achieve a certain treatment effect.

To prevent the wire(s) 22 from potentially damaging the balloon 12 when energy is applied thereto, an insulator 26 may optionally be provided. This may comprise a rigid material, such as a ceramic, extending between the balloon 12 outer surface and a corresponding conductive wire 22, and thus could also aid in enhancing the scoring effect as a result of the rigidity when the balloon is inflated. The insulator 26 could also take the form of a flexible, thin-film insulator. The insulator 26 may comprise a flat upper surface for engaging the corresponding wire 22, or may comprise a U-shaped structure for cradling and capturing the wire 22 against transverse movement.

In any case, the insulator 26 may be attached to the outer surface of the balloon 12 along the cylindrical barrel section 12b, and may have a width in a circumferential direction the same or greater than the diameter of the corresponding wire 22. Alternatively or additionally, the insulator 26 may be attached to the wire 22, such as by an adhesive or other form of bond, such as a tie (which modes of attachment may be optional in the case where the insulator 26 is designed to capture and hold the wire in place).

The portions 22a, 22c of the wire(s) 22 along the proximal and distal tapered cones 12a, 12c may be insulated, such as by an insulating covering or coating. Accordingly, in this arrangement, the application of electrical energy occurs only along the exposed portion 22b of the wire 22 along the working surface S of the catheter 10. In the illustrated embodiment, this exposed portion 22b is coextensive with the cylindrical barrel section 12b.

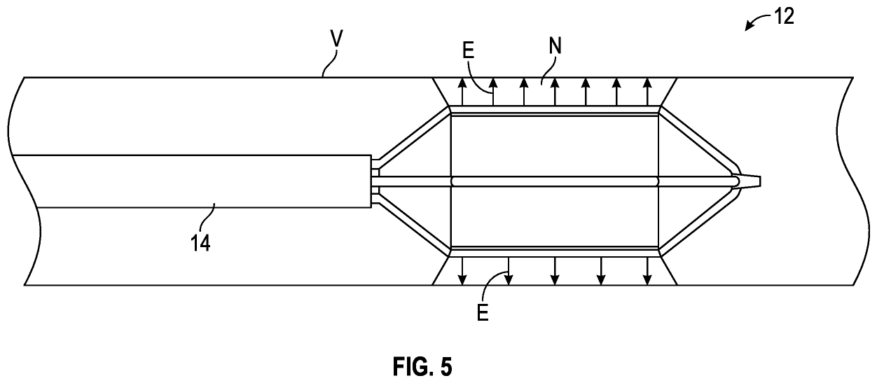

In use, and with reference to FIG. 5, the catheter 10 may be positioned in a vessel V with the balloon 12 using guidewire 30 guidance to track to the desired treatment area, such as adjacent to or within a structure such as a lesion N (and possibly within a sheath (not shown) proximal of the lesion). The balloon 12 once in position may then be at least partially or fully inflated using indeflator 34 such that the scoring wire(s) 22 engage and score the lesion N at one or more locations. At any time before, after, or during the scoring step, electrical energy, may be applied to the wire(s) 22 to provide ablation, such as via radio frequency waves to achieve RF ablation. The result enhances the treatment effect achieved (note radial arrows E to indicate the application of energy from the wire(s) 22 to the lesion N).

As can be appreciated, this can be done without the need for over-pressurizing the balloon 12, since the wire(s) 22 need only contact the lesion to transfer energy thereto. This would minimize overstretch and potential dissections, as the balloon 12 would only need to be inflated to lightly touch the lesion then the wire 22 would provide the treatment effect via ablation. The generator 24 could be used selectively in order to prevent ablation of healthy tissue, such as in an eccentric or irregular lesion.

The above steps for treatment may be repeated in any order as necessary to achieve the desired result, and may be repeated while rotating the balloon 12 within the anatomical structure (i.e., lesion N or vessel V). The steps may also be repeated on other lesions or for anatomical structures at other treatment locations during a single intervention, as desired. At the conclusion of the treatment, the catheter 10 may be withdrawn from the vessel.

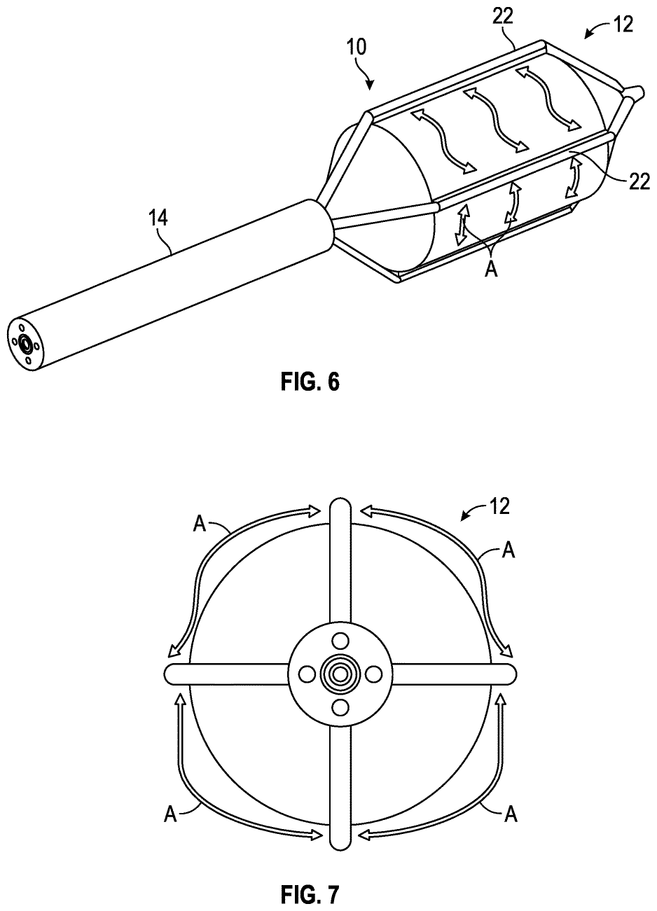

As a further embodiment, the application of electrical energy may involve performing irreversible electroporation (IRE). Thus, as shown in FIGS. 6 and 7, adjacent pairs of wires 22 may work as bipolar electrodes to form an arc or electrical field therebetween (as shown with arrows A) in order to achieve ablation without using thermal energy. This may be useful in more organized clot/thrombus/stenosis (subacute and chronic).

The wire(s) 22 are shown as being elongated along a longitudinal axis of the balloon 12. Different shapes of wire(s) 22 could be used. For example, the wire(s) 22 could zig-zag or follow an S-shaped path along the cylindrical barrel section 12b. A single spirally wrapped wire 22 could also be used. While a wire 22 with a generally circular cross section is shown in FIG. 3A, the wire cross-section could be oval, triangular, rectangular (e.g., a ribbon-shaped wire), or take any other shape, without limitation, and may comprise different shapes along different portions of the wire, if desired.

The conductive material of the wire(s) may be strong and resilient, such as for example Tungsten-Rhenium or similar alloys, but conductive polymers could also be used with sufficient hardness to provide scoring effect. The balloon may comprise conventional balloon materials, such as PET or nylon. The balloon 12 may be non-compliant (which may be achieved by incorporating one or more inelastic fibers) or semi-compliant.

Aside from treating lesions, other possible treatments may be performed using the disclosed catheter 10. For instance, it may be useful for denervation, such as for renal nerves. In such case, the evenly spaced apart wires 22 allow for circumferential treatment of the renal nerves at each longitudinal distance.

In the case of renal stenosis, the catheter 10 could also be used for scoring prior to denervation, which potentially could lead to better clinical outcomes as a result of the direct energy transfer achieved. The generator 24 may also be controlled in this situation to focus energy to specific nerve bundles/areas as needed.

Other possible applications include superficial venous closure, which could benefit from the application of energy afforded. Fusing vessels such as for forming a fistula could also be done, using for example a small diameter balloon to apply RF energy to fuse and create scar tissue in conduit between the two vessels. The catheter 10 may also be used for tumor ablation.

Summarizing, this disclosure relates to the following items:
1. An apparatus for performing a medical procedure on an anatomical structure, comprising:
   an inflatable balloon;
   at least one conductive wire extending along the balloon and adapted for scoring the anatomical structure when the inflatable balloon is inflated; and
   a generator for supplying electrical energy to the at least one conductive wire.
2. The apparatus of item 1, further including an insulator between an outer surface of the inflatable balloon and the at least one conductive wire.
3. The apparatus of item 2, wherein the insulator comprises a ceramic material.
4. The apparatus of item 2 or item 3, wherein the insulator comprises a cradle for receiving the at least one conductive wire.
5. The apparatus of any of items 1-4, wherein the at least one conductive wire comprises a plurality of conductive wires.
6. The apparatus of item 5, wherein the generator is adapted to apply energy selectively to one or more of the plurality of conductive wires.
7. The apparatus of any of items 1-6, further including a shaft supporting the inflatable balloon, the shaft including a lumen for receiving a portion of the at least one conductive wire proximal of the balloon.
8. The apparatus of any of items 1-7, wherein the balloon comprises a tapered proximal section and a barrel section distal thereof, a portion of the at least one conductive wire along the tapered proximal section being insulated.
9. The apparatus of any of items 1-8, wherein the generator is adapted to perform RF ablation.
10. The apparatus of any of items 1-8, wherein the generator is adapted to perform irreversible electroporation.
11. An apparatus for performing a medical procedure on an anatomical structure, comprising:
   an inflatable balloon;
   at least one wire extending along the balloon and adapted for scoring the anatomical structure when the inflatable balloon is inflated; and
   a rigid member between an outer surface of the inflatable balloon and the at least one wire.
12. The apparatus of item 11, wherein the rigid member comprises an insulator.
13. The apparatus of item 10 or item 11, further including a generator for supplying electrical energy to the at least one wire, which is conductive.
14. The apparatus of item 13, wherein the generator is adapted to perform RF ablation.
15. The apparatus of item 13, wherein the generator is adapted to perform irreversible electroporation.
16. The apparatus of any of items 11-15, further including a shaft supporting the inflatable balloon, the shaft including a lumen for receiving a portion of the at least one wire proximal of the balloon.
17. The apparatus of any of items 11-16, wherein the balloon comprises a tapered proximal section and a barrel section distal thereof, a portion of the at least one wire along the tapered proximal section being insulated.
18. The apparatus of any of items 11-17, wherein the rigid member comprises a cradle for receiving the at least one wire.
19. A method of performing a procedure on an anatomical structure, comprising:
   scoring the structure with a scoring wire extending along an inflatable balloon; and
   applying electrical energy to the scoring wire.
20. The method of item 19, wherein the step of applying electrical energy comprises performing RF ablation.
21. The method of item 19, wherein the step of applying electrical energy comprises performing irreversible electroporation.
22. The method of any of items 19-21, further including the step of providing an insulator for insulating the at least one scoring wire from an outer surface of the inflatable balloon.
23. The method of any of items 19-22, wherein the scoring balloon includes a plurality of scoring wires, and the step of applying electrical energy comprises applying electrical energy to each of the plurality of scoring wires simultaneously.
24. The method of any of items 19-22, wherein the scoring balloon includes a plurality of scoring wires, and the step of applying electrical energy comprises selectively applying electrical energy alternatively to the plurality of scoring wires.
25. The apparatus or method of any of items 1-24, wherein the anatomical structure is selected from the group consisting of a vessel, a lesion, or a nerve.
26. A method of performing a medical procedure on an anatomical structure, comprising:
   scoring the structure with a scoring wire spaced from an outer surface of an inflatable balloon by a rigid member.
27. The method of item 26, further including the step of applying electrical energy to the scoring wire.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are ap-propriate to perform in the disclosed invention. However, it is to be understood that the value to which such modifiers refer is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the spirit and scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. An apparatus for performing a medical procedure on an anatomical structure, comprising:
an inflatable balloon;
at least one conductive wire extending along the balloon and adapted for scoring the anatomical structure when the inflatable balloon is inflated; and
a generator for supplying electrical energy to the at least one conductive wire.

2. The apparatus of claim 1, further including an insulator between an outer surface of the inflatable balloon and the at least one conductive wire.

3. The apparatus of claim 2, wherein the insulator comprises a ceramic material.

4. The apparatus of claim 2, wherein the insulator comprises a cradle for receiving the at least one conductive wire.

5. The apparatus of claim 1, wherein the at least one conductive wire comprises a plurality of conductive wires.

6. The apparatus of claim 5, wherein the generator is adapted to apply energy selectively to one or more of the plurality of conductive wires.

7. The apparatus of claim 1, further including a shaft supporting the inflatable balloon, the shaft including a lumen for receiving a portion of the at least one conductive wire proximal of the balloon.

8. The apparatus of claim 1, wherein the balloon comprises a tapered proximal section and a barrel section distal thereof, a portion of the at least one conductive wire along the tapered proximal section being insulated.

9. The apparatus of claim 1, wherein the generator is adapted to perform ablation by applying energy to the wire adapted to generate radiofrequency waves.

10. The apparatus of claim 1, wherein the generator is adapted to perform ablation by applying energy to the at least one conductive wire as a first electrode and a second electrode to perform irreversible electroporation.

11. An apparatus for performing a medical procedure on an anatomical structure, comprising:
an inflatable balloon;
at least one scoring wire extending along the balloon and adapted for scoring the anatomical structure when the inflatable balloon is inflated; and
a rigid member between an outer surface of the inflatable balloon and the at least one scoring wire.

12. The apparatus of claim 11, wherein the rigid member comprises an insulator.

13. The apparatus of claim 11, wherein the scoring wire is conductive, and further including a generator for supplying electrical energy to the at least one scoring wire.

14. The apparatus of claim 13, wherein the generator is adapted to perform RF ablation.

15. The apparatus of claim 13, wherein the generator is adapted to perform irreversible electroporation.

16. The apparatus of claim 11, further including a shaft supporting the inflatable balloon, the shaft including a lumen for receiving a portion of the at least one scoring wire proximal of the balloon.

17. The apparatus of claim 11, wherein the balloon comprises a tapered proximal section and a barrel section distal thereof, a portion of the at least one scoring wire along the tapered proximal section being insulated.

18. The apparatus of claim 11, wherein the rigid member comprises a cradle for receiving the at least one scoring wire.

19. A method of performing a procedure on an anatomical structure, comprising:
scoring the structure with a scoring wire extending along an inflatable balloon; and
applying electrical energy to the scoring wire.

20. The method of claim 19, wherein the step of applying electrical energy comprises performing RF ablation.

21-27. (canceled)
